(19) United States
(12) Patent Application Publication
(10) Pub. No.: US 2022/0280471 A1
(43) Pub. Date: Sep. 8, 2022

(54) Treatment of Affective Disorders

(71) Applicant: Vanda Pharmaceuticals Inc., Washington, DC (US)

(72) Inventors: Mihael Polymeropoulos, Potomac, MD (US); Christos Polymeropoulos, Potomac, MD (US); Changfu Xiao, Vienna, VA (US)

(21) Appl. No.: 17/804,126

(22) Filed: May 26, 2022

Related U.S. Application Data
(63) Continuation of application No. 16/636,292, filed on Feb. 3, 2020, now abandoned.
(60) Provisional application No. 62/540,200, filed on Aug. 2, 2017.

(51) Int. Cl. A61K 31/343; A61P 25/24; A61K 9/00

(52) U.S. Cl. CPC A61K 31/343; A61P 25/24; A61K 9/0053

(57) ABSTRACT

Embodiments of the invention relate generally to the treatment of affective disorders and, more particularly, to the treatment of affective disorders in a patient of Majority Black African ancestry.

### CROSS-REFERENCE TO RELATED APPLICATIONS

This application claims the benefit of co-pending U.S. patent application Ser. No. 16/636,292, filed 3 Feb. 2020, which is the US national phase of PCT Patent Application Ser. No. PCT/US18/44995, filed 2 Aug. 2018, which claims priority to then-co-pending U.S. Provisional Patent Application Ser. No. 62/540,200, filed 2 Aug. 2017, each of which is hereby incorporated herein as though fully set forth.

### BACKGROUND

Affective disorders are mood disorders and include, among other disorders, depressive disorders. Illustrative affective disorders include attention deficit hyperactivity disorder (ADHD), bipolar disorder, body dysmorphic disorder, bulimia nervosa and other eating disorders, cataplexy, dysthymia, generalized anxiety disorder, hypersexuality, irritable bowel syndrome (IBS), impulse-control disorders, kleptomania, migraine, major depressive disorder (MDD), obsessive-compulsive disorder (OCD), oppositional defiant disorder, panic disorder, post-traumatic stress disorder (PTSD), premenstrual dysphoric disorder, social anxiety disorder, and fibromyalgia.

In addition, other disorders may be part of a spectrum accompanying or associated with affective disorders. These include, for example, chronic pain, intermittent explosive disorder, pathological gambling, personality disorder, pyromania, substance abuse and addiction including alcoholism, and trichotillomania.

Depressive disorders affect nearly 20 million adults in the US and are characterized by an array of symptoms, including persistent sad, anxious, or empty mood; feelings of hopelessness or pessimism; feelings of guilt, worthlessness, or helplessness; loss of interest or pleasure in hobbies and activities that were once enjoyed, including sex; decreased energy, fatigue, or a feeling of being “slowed down;” difficulty concentrating, remembering, or making decisions; insomnia, early-morning awakening, or oversleeping; appetite and/or weight loss; overeating and weight gain; thoughts of death or suicide; suicide attempts; restlessness or irritability; and persistent physical symptoms that do not respond to treatment, such as headaches, digestive disorders, and chronic pain. Untreated depressive disorders may be both emotionally and physically debilitating.

The National Institute of Mental Health (NIMH) has identified three of the most common types of depressive illness as: Major depressive disorder (MDD), dysthymia, and bipolar disorder (also referred to as manic-depressive illness).

MDD is manifested by a combination of symptoms that interfere with one's ability to work, study, sleep, eat, and enjoy once-pleasurable activities. MDD may occur only once, but more commonly occurs several times during a patient's lifetime. MDD is often associated with insomnia and it has been hypothesized that circadian rhythm disruption may be involved in the etiology of MDD.

Dysthymia is typically less severe than MDD and involves long-term, chronic symptoms that do not disable but prevent one from functioning well or feeling well. Patients with dysthymia may also experience episodes of MDD at times.

Bipolar disorder is not as common as other depressive disorders and is characterized by mood changes between severe highs (manic episodes) and severe lows (depressive episodes). Mood changes may be rapid and dramatic, but are more typically gradual. During a depressive episode, an individual may experience any or all of the symptoms of other depressive disorders. During a manic episode, an individual may be overactive, overly talkative, and have a lot of energy. Manic episodes often affect thinking, judgment, and social behavior that result in embarrassment or other problems. Untreated, manic episodes may worsen to a psychotic state.

Tasimelteon (trans-N-[[2-(2,3-dihydrobenzofuran-4-yl)cycloprop-1yl]methyl] propanamide) is a circadian regulator which binds specifically to two high affinity melatonin receptors, Mel1a (MT1R) and Mel1b (MT2R). These receptors are found in high density in the suprachiasmatic nucleus of the brain (SCN), which is responsible for synchronizing our sleep/wake cycle.

Tasimelteon is disclosed in U.S. Pat. No. 5,856,529 and US Patent Application Publication No. 2009/0105333. Tasimelteon improves sleep parameters in clinical studies which simulate a desynchronization of the circadian clock. Tasimelteon has so far been studied in hundreds of individuals and is shown a good tolerability profile.

The use of tasimelteon in treating depressive disorders is described in US Patent Application Publication No. 2009/0209638.

The use of tasimelteon in entraining the circadian rhythm of an individual to a 24-hour period is described in U.S. Pat. No. 8,785,492.

The effects of CYP1A2 inhibitors and CYP3A4 inhibitors on the administration of tasimelteon are described in US Patent Application Publication No. 2015/0025086.

The effects of food on the bioavailablity of tasimelteon are described in US Patent Application Publication No. 2015/0196527.

An individual with genetic “Majority Black African” ancestry, as used in this specification, refers to an individual having ancestry in any of the black African populations that is about 50% or greater black African ancestry. This definition encompasses individuals, for example, with about 50% or 70% or greater West African ancestry. African-Americans, as that term is used here, is defined as a subpopulation of Americans who are “Majority Black African.” In this regard, Bryc et al., for example, report that, on average, African-American populations comprise 73.2-82.1% West African, 16.7-24% European, and 0.8-1.2% Native American genetic ancestry, although there is large individual variation.

### SUMMARY

Embodiments of the invention relate generally to the treatment of an affective disorder in individuals of Majority Black African genetic ancestry, including African-Americans by administering to the patient an amount of tasimelteon effective to treat such disorder.

According to various embodiments of the invention, tasimelteon is administered: at a dose of 20 mg/day, prior to bedtime (e.g., 0.5 hour to 2 hours prior), under fasted conditions, while avoiding the coadministration of rifampicin, while avoiding the co-administration of fluvoxamine, while avoiding administration to a patient who smokes, to a patient having high or low endogenous melatonin, or any combination thereof.

In some embodiments of the invention, the treatment of an affective disorder in a Majority Black African (including African-American) patient includes entraining the patient's circadian rhythm, melatonin rhythm, and/or cortisol rhythm to a 24-hour sleep-wake cycle by administering to the patient an effective amount of tasimelteon.

### DETAILED DESCRIPTION

A large clinical study was conducted to evaluate the effects of tasimelteon on depressive symptoms in patients with MDD. Details of the study design are set out in study number NCT01428661, available at clinicaltrials.gov. Aspects of the study relevant to the claimed invention are described below.

A total of 507 patients with MDD were enrolled in a double-masked, placebo-controlled study randomized on either tasimelteon 20 mg (n=254) or placebo (n=253). Patients were assessed at baseline and weekly for eight weeks on a number of depression scales, including the Hamilton Depression Scale (HAMD).

Tasimelteon- and placebo-treated patients appeared to improve similarly from baseline by 8.1 and 7.8 points, respectively, on the HAMD scale (p=0.57). An analysis by reported race, however, reveals a significant positive effect of tasimelteon among African-American patients. Of the 507 randomized patients, 166 (32.7%) are African-American, of which 78 received tasimelteon and 88 placebo. A majority of the remaining patients included in the study are Caucasian.

At eight weeks, African-American MDD patients treated with tasimelteon show improvement by 9.9 points on the HAMD scale, as compared to 6.9 points for the placebo-treated patients (p=0.018). In a responder analysis (improvement in the HAMD scale of 50% or greater from baseline), 59% of tasimelteon-treated show improvement, as compared to 30% of placebo-treated patients (p=0.0019).

These results suggest that tasimelteon 20 mg improves symptoms of depression in African-American patients with MDD. These results also support the suggestion noted above of a circadian component in the etiology and treatment of MDD.

In addition, among the study subjects, melatonin onset (MO) occurs significantly (p=0.0229) earlier among African-American MDD patients than Caucasian MDD patients. In African-American MDD patients, mean MO occurs at 21:07±1:53 hours (range 14:58-01:50 hours). In Caucasian MDD patients, mean MO occurs at 21:36±1:53 hours (range 16:23-03:54 hours). This additional observation may help to explain the improved efficacy in African-Americans as compared to non-African-American participants in the study.

Embodiments of the invention therefore include the treatment of an affective disorder in a Majority Black African patient in need of such treatment. In one embodiment, the invention includes treating an affective disorder in a Majority Black African patient by administering to the patient an amount of tasimelteon effective to treat said affective disorder.

Such administration, according to some embodiments, is prior to bedtime, e.g., between about 0.5 hour and about 2 hours prior to a target bedtime.

According to some embodiments, the amount of tasimelteon effective to treat the affective disorder is between 10 mg/day and 100 mg/day, e.g., between 20 mg/day and 50 mg/day, e.g., 20 mg/day.

Other embodiments include entraining a circadian rhythm, a cortisol rhythm, or a melatonin rhythm of a Majority Black African patient to a 24-hour sleep-wake cycle by orally administering to the patient 20 mg of tasimelteon once daily before a target bedtime. In such embodiments, a 24-hour sleep-wake cycle is a cycle in which the patient goes to sleep at or near a target bedtime and awakens at or near a target wake time following a daily sleep period of approximately 7 to 9 hours.

These and other embodiments may include administering tasimelteon to a Majority Black African patient after discontinuing the administration of fluvoxamine or rifampicin to said patient, administering tasimelteon to such patient after such patient discontinues smoking, and/or administering tasimelteon to such patient without food.

What is claimed is:

1. A method of treating a Majority Black African patient suffering from an affective disorder comprising:
administering to said patient an amount of tasimelteon effective to treat said affective disorder,
wherein the affective disorder is selected from a group consisting of: attention deficit hyperactivity disorder (ADHD), bipolar disorder, body dysmorphic disorder, bulimia nervosa and other eating disorders, cataplexy, dysthymia, generalized anxiety disorder, hypersexuality, irritable bowel syndrome (IBS), impulse-control disorders, kleptomania, migraine, major depressive disorder (MDD), obsessive-compulsive disorder (OCD), oppositional defiant disorder, panic disorder, post-traumatic stress disorder (PTSD), premenstrual dysphoric disorder, social anxiety disorder, and fibromyalgia.

2. The method of claim 1, wherein the affective disorder is a depressive disorder.

3. The method of claim 1, wherein the affective disorder is major depressive disorder (MDD).

4. The method of claim 1, wherein the amount of tasimelteon is between 10 mg/day and 100 mg/day.

5. The method of claim 5, wherein the amount of tasimelteon is between 20 mg/day and 50 mg/day.

6. The method of claim 6, wherein the amount of tasimelteon is 20 mg/day.

7. The method of claim 1, wherein administering includes administering prior to bedtime.

8. The method of claim 7, wherein administering includes administering between about 0.5 hour and about 2 hours prior to a target bedtime.

9. The method of claim 1, wherein administering includes administering under fasted conditions.

10. The method of claim 1, further comprising:
administering to the patient a second antidepressant medication.

11. The method of claim 1, wherein the patient exhibits at least one symptom selected from a group consisting of: persistent sad, anxious, or empty mood; feelings of hopelessness; pessimism; feelings of guilt, worthlessness, or helplessness; loss of interest or pleasure in hobbies and activities that were once enjoyed, including sex;
decreased energy, fatigue, or being slowed down; difficulty concentrating, remembering, or making decisions; insomnia, early-morning awakening, or oversleeping; appetite and/or weight loss or overeating and weight gain; thoughts of death or suicide; suicide attempts; restlessness; irritability; persistent physical symptoms that do not respond to treatment, including headaches, digestive disorders, and chronic pain; and any combination of the preceding.

12. A method of entraining to a 24-hour sleep-wake cycle in which the patient goes to sleep at or near a target bedtime and awakens at or near a target wake time following a daily sleep period of approximately 7 to 9 hours: a circadian rhythm, a cortisol rhythm, and/or a melatonin rhythm of a Majority Black African patient suffering from an affective disorder, the method comprising:
orally administering to the patient 20 mg of tasimelteon once daily before a target bedtime,
wherein the affective disorder is selected from a group consisting of:
attention deficit hyperactivity disorder (ADHD), bipolar disorder, body dysmorphic disorder, bulimia nervosa and other eating disorders, cataplexy, dysthymia, generalized anxiety disorder, hypersexuality, irritable bowel syndrome (IBS), impulse-control disorders, kleptomania, migraine, major depressive disorder (MDD), obsessive-compulsive disorder (OCD), oppositional defiant disorder, panic disorder, post-traumatic stress disorder (PTSD), premenstrual dysphoric disorder, social anxiety disorder, and fibromyalgia.

13. The method of claim 12, wherein the affective disorder is a depressive disorder.

14. The method of claim 13, wherein the affective disorder is major depressive disorder (MDD).

15. The method of claim 12, wherein the tasimelteon is administered 0.5 hour to 1.5 hours before the target bedtime.

16. In a method of administering tasimelteon to a patient, the improvement comprising selecting as such patient a Majority Black African patient suffering from an affective disorder selected from a group consisting of: attention deficit hyperactivity disorder (ADHD), bipolar disorder, body dysmorphic disorder, bulimia nervosa and other eating disorders, cataplexy, dysthymia, generalized anxiety disorder, hypersexuality, irritable bowel syndrome (IBS), impulse-control disorders, kleptomania, migraine, major depressive disorder (MDD), obsessive-compulsive disorder (OCD), oppositional defiant disorder, panic disorder, post-traumatic stress disorder (PTSD), premenstrual dysphoric disorder, social anxiety disorder, and fibromyalgia.

17. The improvement of claim 16, wherein the affective disorder is a depressive disorder. 20

18. The improvement of claim 17, wherein the affective disorder is major depressive disorder (MDD).

19. The improvement of claim 18, wherein administering tasimelteon to the patient includes orally administering 20 mg of tasimelteon once daily between about 0.5 hour and about 2 hours prior to a target bedtime.

20. The improvement of claim 19, wherein administering tasimelteon includes administering the tasimelteon under fasted conditions.
